(19) United States
(12) Patent Application Publication
(10) Pub. No.: US 2010/0021498 A1
(43) Pub. Date: Jan. 28, 2010

(54) LIVE, ATTENUATED PNEUMOCOCCAL VACCINE

(76) Inventor: Jeffrey Weiser, Merion, PA (US)

Correspondence Address: Pearl Cohen Zedek Latzer, LLP, 1500 Broadway, 12th Floor, New York, NY, 10036, US

(21) Appl. No.: 12/281,145

(22) PCT Filed: Mar. 28, 2007
(86) PCT No.: PCT/US2007/007676

§ 371 Date: Jan. 30, 2009

Related U.S. Application Data
(60) Provisional application No. 60/786,351, filed on Mar. 28, 2006.

(51) Int. Cl. A61K 39/09; C12N 1/20; C12N 15/87

(52) U.S. Cl. 424/203.1; 435/253.4; 424/244.1; 435/440

(57) ABSTRACT

This invention relates to a live mutated strain of S. pneumoniae which is incapable of expressing polysaccharide capsule, while still capable of colonizing the nasopharynx of the subject.

### FIELD OF INVENTION

This invention is directed to a vaccine for protecting a subject against pneumococcal infection. Specifically, the invention is directed to a live mutated strain of S. pneumoniae which is incapable of expressing polysaccharide capsule, while still capable of colonizing the nasopharynx of the subject.

### BACKGROUND OF THE INVENTION

The pneumococcus still ranks among the leading infectious causes of morbidity and mortality throughout the world. It is estimated that pneumococcal pneumonia is responsible for over 40,000 deaths per year in the US (predominantly in the elderly) and 1,000,000 per year worldwide (predominantly in young children). Other common infections frequently caused by this organism include acute otitis media, chronic bronchitis, acute sinusitis, and meningitis. (Otitis media is the most common reason for children to receive medical attention in this country and S. pneumoniae is the leading bacterial cause.) Much of the morbidity and mortality associated with yearly outbreaks of influenza is caused by secondary pneumococcal pneumonia. Concerns for pandemic influenza have heightened the potential for increased pneumococcal disease.

The pneumococcus has proven to be a particularly adaptable foe. Optimism concerning the treatment of pneumococcal infection that followed the development of penicillin and other classes of antimicrobials has faded due the acquisition and widespread dissemination of resistance. The growing problem of antibiotic resistance has emphasized the need for prevention. A vaccine consisting of 23 of the 90 known capsular polysaccharides (PnPS) produced by this species is used in adults but its overall impact remains controversial. The vaccine is not effective in young children as they respond to type 2, T cell-independent polysaccharide antigens without class switching to produce effective IgG or a memory response. In 2000, a conjugate vaccine (PRENEVAR™), which induces T-dependent responses to PnPSs was licensed in the US and has been effective in children at preventing invasive disease caused by the seven types in its formulation. This vaccine was the first to exceed a billion dollar/yr in sales and estimates predict an annual market of over $3,000,000,000 by 2010. The immune response to this systemically administered vaccine diminishes carriage resulting in herd immunity that has also had a significant impact on the incidence of disease in unvaccinated adults. This experience has confirmed that young children are the major reservoir for this organism.

There are, however, several major limitations of the conjugate vaccine. Its effectiveness against the most frequent manifestations of infection-mucosal infection (pneumonia and otitis) seems to far more limited than for invasive disease. The cost of this complex product is prohibitive for populations in greatest need. This is especially unfortunate since recent data from the Gambia shows that it could have a major impact on childhood pneumonia and overall mortality in the developing world. The vaccine requires multiple injections.

Increasing rates of resistance of S. pneumoniae to antibiotics highlight the priority of preventing pneumococcal disease. There are currently two commercially available vaccines against S. pneumoniae, both of which are based on the polysaccharide capsule, which is the major determinant necessary for causing disease. Pneumococcal isolates express at least 90 structurally unique capsular polysaccharides, its serotype determining antigen. Pneumovax© is a 23-valent polysaccharide vaccine, which contains the most common types causing pneumococcal infection, and is effective in adults, but not in early childhood. Prevnar© is a 7-valent polysaccharide conjugate vaccine (containing types 4, 6B, 9V, 14, 18C, 19F, and 23F), which is effective in young children, but has been shown to induce selective pressure and the gradual replacement with non-vaccine types (serotype replacement). Moreover, its effectiveness against the most frequent manifestations of infection, mucosal infection (pneumonia and otitis), seems far more limited than for invasive disease, and the conjugate vaccine is complex and costly, making it inaccessible for populations in greatest need. It is therefore desirable to find new and innovative strategies for vaccine development

### SUMMARY OF THE INVENTION

In one embodiment, the invention provides a mutated strain derived from a parent Streptococcus pneumoniae strain, wherein said cells exhibit attenuated pathogenicity compared to those of the parent strain, and wherein the mutation is in cps, ply, pspA or their combination.

In another embodiment, the invention provides a vaccine for treating, preventing or ameliorating a subject against pneumococcal infection or colonization, comprising a pharmaceutically acceptable carrier and an immunologically effective amount of mutated strain derived from a parent Streptococcus pneumoniae strain, wherein the mutation is in cps, ply, pspA or their combination.

In one embodiment, the invention provides a method of protecting a subject against disease or colonization by a Streptococcus pneumoniae strain, comprising administering to said subject a composition comprising an immunologically effective amount of live cells of a mutated strain derived from a parent Streptococcus pneumoniae strain, wherein said cells exhibit attenuated pathogenicity compared to those of the parent strain, and wherein the mutation is in cps, ply, pspA or their combination.

In another embodiment, the invention provides a method for preparing a mutated strain of a species of S. pneumoniae for use in a vaccine for protecting a subject against pneumococcal infection or colonization, comprising the steps of: selecting a pathogenic parent S. pneumoniae strain capable of effectively colonizing the subject's nasopharynx; deleting an entire operon of the gene encoding pneumolysin (ply), pneumococcal surface protein A (pspA), capsular polysaccharide (cps), or their combination in the selected strains, wherein the entire operon is deleted from strains that are spontaneously resistant to a predetermined antibiotic; replacing the entire operon with an operon containing the desired mutation, or double mutation; and knocking out genetic exchange, thereby preventing reversion or loss of the attenuating mutation in the mutated strain, and thereby obtaining a mutated strain, wherein said cells exhibit attenuated pathogenicity compared to those of the parent strain, and wherein said cells are capable of triggering an immune response that protects the subject against pneumococcal infection when administered as a live vaccine.

In one embodiment, the invention provides a method for preparing a vaccine for preventing or protecting a subject against pneumococcal infection or colonization, comprising the steps of selecting a pathogenic parent S. pneumoniae strains capable of effectively colonizing the subject's nasopharynx; deleting an entire operon of the gene encoding pneumolysin (ply), pneumococcal surface protein A (pspA), capsular polysaccharide (cps), or their combination in the selected strains, wherein the entire operon is deleted from strains that are spontaneously resistant to a predetermined antibiotic; replacing the entire operon with an operon containing the desired mutation, or double mutation; knocking out genetic exchange, thereby preventing reversion or loss of the attenuating mutation in the mutated strain, and combining the cells containing the desired mutation with a pharmaceutically acceptable carrier in a form suitable for administration as a live vaccine to the subject.

In another embodiment, the invention provides a method of vaccinating a subject against pneumococcal infection or colonization, comprising the step of administering to the subject an immunologically effective amount of a vaccine and a pharmaceutically acceptable carrier, wherein said vaccine comprises a mutated strain derived from a parent Streptococcus pneumoniae strain, wherein said cells exhibit attenuated pathogenicity compared to those of the parent strain, and wherein the attenuating mutation is in cps, ply, pspA or their combination, and wherein said cells are capable of triggering an immune response that protects the subject against pneumococcal infection or colonization when administered as a live vaccine.

### SHORT DESCRIPTION OF THE DRAWINGS

FIG. 1. Effect of pneumococcal capsule on the density of nasal colonization. Two days following intranasal challenge with 10⁷ CFU of an isolate of the type indicated (gray bars) or its defined unencapsulated mutant (black bars) lacking the entire cps locus, C57BL/6 mice were sacrificed for quantitative culture of upper respiratory tract lavage fluid. Values represent the mean of 5-10 mice/group±SD. Dashed line represents limit of detection. * P<0.05,**P<0.01 compared to encapsulated parent strain (Mann-Whitney).

FIG. 2. Effect of restoration of encapsulation on the density of colonization. Unencapsulated TIGR4 cps- (black bar) or transformants with the cps locus of the type indicated (open bars) that corrected the loss of capsule expression were compared for their ability to colonize mice at 2 days post-inoculation. Acquisition of the type indicated was confirmed by quelling. For encapsulated transformants of types 6A and 14 both opaque (hatched bars) and transparent (stippled bars) variants were tested. Dashed line represents limit of detection. Values represent the mean of 5-10 mice/group±SD. * P<0.05 compared to encapsulated transformant (Kruskal-Wallis test with Dunn's post-test for multiple comparisons). ** P<0.03 compared to homotypic transparent variant (Mann-Whitney).

FIG. 3. Time course of the effect of capsule on the density of colonization of strain TIGR4. Following intranasal challenge with 10⁷ CFU of TIGR4 (closed diamonds) or TIGR4 cps- (open squares), the density of colonization was assessed in quantitative culture of upper respiratory tract lavage fluid on the day post-inoculation indicated. Dashed line indicates the limit of detection. Values represent the mean of 5-10 mice/strain at each time point±SD. * P<0.03 compared to unencapsulated mutant at the same time point (Mann-Whitney).

FIG. 4. Effect of neutrophils, complement and functional antibody on colonization by an unencapsulated mutant. C57Bl/6 mice were pretreated with RB6-8C5 to deplete neutrophils (or rat IgG control) or cobra venom factor (CoVF) to deplete complement (or vehicle control) prior to intranasal challenge with 10⁷ CFU of TIGR4 cps- and the density of colonization assessed in quantitative culture of upper respiratory tract lavage fluid on post-inoculation day 2. Colonization of congenic μMT mice was determined in parallel experiments. Values represent the mean of 5-10 mice/strain at each time point±SD. P>0.05 (Kruskal-Wallis test with Dunn's post-test for multiple comparisons).

FIG. 5. Early events in colonization showing transition from mucus to the epithelial surface for encapsulated pneumococcal strain TIGR4. □ Frozen nasal tissue from C57 Bl/6 mice colonized for 30 minutes (A) or 2 days (B) and stained with type-specific sera detected with Cy3 secondary antibody (red) and DAPI (blue) superimposed with Normarski brightfield optics to show the epithelial border. Frozen nasal tissue from C57 Bl/6 mice colonized 30 minutes (C) or 2 days (D) stained with type-specific sera detected with HRP-conjugated secondary antibody and DAB substrate (brown), alcian blue (pH 2.5) (blue) and nuclear fast red (red). Arrows indicate bacteria. 10 μm scale bar.

FIG. 6. Unencapsulated pneumococci remain trapped within lumenal mucus. H&E stained frozen nasal tissue from C57 Bl/6 mice 30 minutes post-inoculation with (A) TIGR4 or (B) TIGR4 cps-. Only the unencapsulated mutant is agglutinated in mucoid material in the lumen. Frozen nasal tissue from C57 Bl/6 mice 20 hours post-inoculation with (C) TIGR4 or (D) TIGR4 cps- stained with type-specific sera detected with Cy3 secondary antibody (red) and DAPI (blue). Tissue auto-fluorescence (green) reveals the epithelial border. 10 μm scale bar.

FIG. 7. Systemic and mucosal protection by a live, attenuated pneumococcal vaccine. (A) Kaplan-Meier survival plot. 10⁷ CFU of P303 cps- (dashed line), or vehicle control, (solid line) was applied intranasally, the mice were allowed to clear their carrier state over 5-6 weeks, and then challenged intranasally with 10⁷ CFU serotype 6A P303. (B) Effect of immunization with P303 cps- or vehicle control on the mean density of colonization±S.D. in surviving mice at 9 days following intranasal challenge with 10⁷ CFU of P303 (grey bars) or TIGR4 (open bars). P<0.02 (Mann-Whitney).

FIG. 8. Low-magnification examination of the distribution of colonizing pneumococci in a murine model over time course of stable carriage. BALB/c mice were given an intranasal dose of 10⁶ CFU of a type 23F isolate P1121 and sacrificed at the time indicated. Frozen tissues on one side of the nasal septum was stained with pneumococcal typing sera followed by Cy3 secondary antibody (red) and DAPI (blue). Composite images taken at 100×.

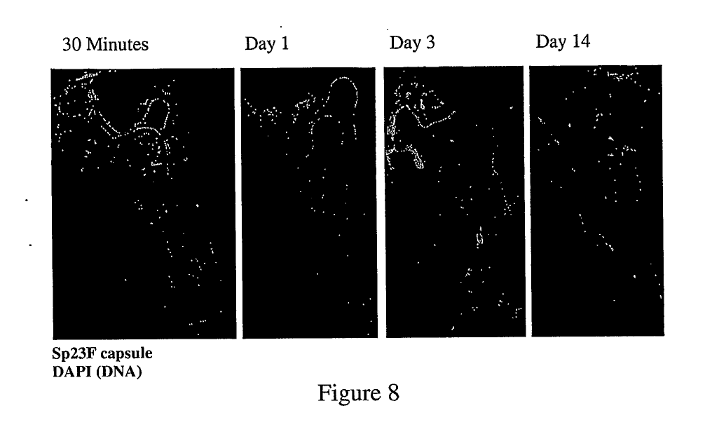

FIG. 9. Systemic and mucosal protection by a live, attenuated pneumococcal vaccine. (A) Kaplan-Meier survival plot. 10⁵⁻⁶ CFU of P303ply-,pspA- (dashed line), or vehicle control, (solid line) was applied intranasally, the mice were allowed to clear their carrier state over 5-6 weeks, and then challenged intranasally with 10⁵ CFU serotype 6A P303. (B) Effect of immunization with P303ply-,pspA- or vehicle control on the mean density of colonization±S.D. in surviving mice at 9 days following intranasal challenge with P303 at the indicated dose (grey bars) or TIGR4 (open bars) 10⁷ CFU. P<0.02 (Mann-Whitney).

FIG. 10. Attenuation of S. pneumoniae: comparison of the level of attenuation of various S. pneumoniae type 6A live vaccines over a 9-day period postinoculation. Data are based on a minimum of 10 animals in each group. Statistical differences compared to the parent strain were determined by the Kaplan-Meier log-rank test.

FIG. 11. Colonization of attenuated S. pneumoniae: comparison of the abilities of various live vaccine strains to colonize the mouse nasopharynx at day 9 (A) and day 2 (B) postinoculation. Density of pneumococci in upper respiratory tract lavage is shown by the mean log CFU/ml±the standard error of the mean. n_{—}4 to 9 mice per group per time point. The dotted line indicates the limit of detection. Statistical differences were determined using the Mann-Whitney test with comparison to the corresponding parent strain. *, P<0.0286; **, P=0.004; ***, P=0.0008.

FIG. 12. Protection induced by live attenuated vaccine strains. (A) Survival rates of C57BL/6 mice immunized with 6Aply/pspA (n=8), 6Acps (n=15), or vehicle-only control (n=14) following intranasal challenge with the 6A parent strain. Statistical differences compared to the vehicle control were determined by the Kaplan-Meier log-rank test. (B) Colonization density at day 9 postinoculation of mice challenged with the type 6A parent strain (solid bars) or type 4 parent strain (hatched bars) after immunization with the indicated live vaccine. The 6Acps/ply vaccine was delivered in two doses prior to challenge. Values represent the means of 6 to 14 mice/group 4 the standard errors of the means. The dotted line indicates the limit of detection. Colonization density was analyzed in surviving mice. Eight mice in the mock-immunized group and one animal in the 6Acps group that did not survive were not included in the analysis. Statistical differences were determined using the Mann-Whitney test with comparisons to the corresponding mock-immunized group. **, P<0.0083.

FIG. 13. Cross-protection induced by live attenuated vaccine strains. (A) Survival rates of C57BL/6 mice immunized with 4 cps in a single dose (n=12) versus two doses (n=12), compared to the vehicle-only control (n=15) following intranasal challenge with the type 6A isolate. Statistical differences compared to the vehicle control were determined by the Kaplan-Meier log-rank test. (B) Colonization density at day 9 postinoculation of mice challenged with the type 4 parent isolate (solid bars) or type 6A parent isolate (hatched bars) after immunization with the indicated live vaccine of the other serotype. One dose of the vaccine strain was administered except where a second dose is indicated (2×). Values represent the means of 6 to 12 mice/group±the standard errors of the means. The dotted line indicates the limit of detection. Colonization density was analyzed in surviving mice. Eight mice in the mock-immunized group and four animals in the 4 cps single-dose group that did not survive were not included in the analysis. Statistical differences were determined using the Mann-Whitney test with comparisons to the corresponding mock-immunized group. *, P<0.0278; **, P=0.0028.

FIG. 14. ELISA titers of IgG and IgA: levels of IgG in serum against type 6A (A) or type 4 (B) (whole bacteria) from mice immunized with the indicated live vaccine. Values shown are the GMT±standard errors of the means (SEMs) following one or two (2×) doses of the vaccine strain. Symbols in panel A: *, P=0.0293; **, P<0.01. Symbols in panel B: *, P=0.0121; **, P=0.007. (C) Levels of IgA against type 6A (whole bacteria) in nasal wash samples from mice immunized with the indicated live vaccine. Values shown are GMT/μg total protein±SEM. *, P<0.02. Statistical differences were determined using the Mann-Whitney test, with comparisons to the mock-immunized control group.

FIG. 15. Mechanism of protection. (A) Survival rates of μMT mice (n=10), MHC II^{-/-} mice (n=8), and wild-type C57BL/6 mice (n=15) immunized with 6Acps, PBS mock-immunized μMT (n=7), or PBS mock-immunized C57BL/6 mice (n=14) and challenged with the 6A parent strain. (B) Colonization density at day 9 postinoculation of mice challenged with the 6A parent strain after immunization with 6Acps. Values represent the means of 8 to 15 mice/group±the standard errors of the means. Statistical differences were determined using the Mann-Whitney test with comparisons to the wild-type C57BL/6 mice.

### DETAILED DESCRIPTION OF THE INVENTION

This invention relates in one embodiment to a live mutated strain of S. pneumoniae which, in one embodiment, is incapable of expressing polysaccharide capsule, while still capable of colonizing the nasopharynx of the subject.

In one embodiment, only encapsulated organisms are able to transit efficiently from their initial site in a host, the lumenal mucus, to the epithelial surface. The capacity of encapsulated, but not unencapsulated, pneumococci to escape from lumenal mucus allow in one embodiment to access host cell receptors beneath the glycocalyx layer covering the tissues of the nasal mucosa. Since only bacteria along these epithelial surfaces demonstrate stable colonization, in another embodiment, escape from the mucus is an important step in persistence and may account for the contribution of encapsulation to the virulence of the infection. In another embodiment, histologic events during early colonization are most consistent with the capsule enhancing escape of the organism from mucus and evasion of clearance by mucociliary flow.

In another embodiment, unencapsulated mutants are avirulent, and may serve as a safe live, attenuated vaccine. According to this aspect of the invention, and in one embodiment, the invention provides a mutated strain derived from a parent Streptococcus pneumoniae strain, wherein said cells exhibit attenuated pathogenicity compared to those of the parent strain, and wherein the mutation is in cps, ply, pspA or their combination.

In one embodiment, the term “attenuated” refers in one embodiment to a cell, culture, or strain of Streptococcus exhibiting a detectable reduction in infectivity or virulence in vitro or in vivo as compared to that of the parent strain of Streptococcus from which the attenuated cell, culture, or strain is derived. Reduction in virulence encompasses any detectable decrease in any attribute of virulence, including infectivity in vitro or in vivo, or any decrease in the severity or rate of progression of any clinical symptom or condition associated with infection.

The term “parent strain” refers in another embodiment, to a strain of Streptococcus which exhibits a relatively higher degree of pathogenicity when administered to a subject than an attenuated strain which is derived therefrom by one or more passages in vivo or in vitro and/or one or more attenuation steps.

Capsular polysaccharides refer in one embodiment, to polysaccharide that is composed of a tetrasaccharide repeating unit containing D-glucose, N-acetyl-D-glucosamine, and D-galactose. An important feature of S. pneumoniae is its capacity to produce a polysaccharide capsule, which is structurally distinct for each of the 90 known serotypes of the organism. In one embodiment, fresh isolates from patients with pneumococcal infection are encapsulated, and spontaneous nonencapsulated (rough) derivatives of such strains are almost completely avirulent. In another embodiment, the capsule comprise S. pneumoniae's type-determining or major antigenic determinant and the basis of all current pneumococcal vaccines. In another embodiment, components other than aldoses and hexoses and their glycopeptidic conjugates comprise the capsular polysaccharide. Those are choline in one embodiment, or other components in other embodiment. In one embodiment, the mutated strain used in the cells, vaccines and methods described herein carries an attenuating mutation resulting in the null expression of capsular polysaccharide and the components expressed therein.

In one embodiment, the S. pneumoniae genes required for biosynthesis and expression of CPS are closely linked on the pneumococcal chromosome. In another embodiment, the locus consists of a central region encoding enzymes for the biosynthesis of the CPS itself, flanked by regions encoding proteins involved in translocation of the CPS across the cytoplasmic membrane (proteins similar to members of the ABC superfamily of ATP-dependent transport proteins) and transport through the cell surface. In one embodiment, the mutated cells, vaccines and methods described herein use genetically-modified strains containing defined mutations in genes in the capsulation locus (cps) but which colonize the nasopharynx at relatively low efficiency. In one embodiment, the mutated strain used in the vaccine, cells and methods described herein, is one in which mutation to the cps gene result in null expression of the capsular polysaccharide and in the mutated strain being unencapsulated.

In one embodiment, PspA is a highly immunogenic antigen that is present on the surface of all S. pneumoniae strains, and although variability exists, in another embodiment, all PspAs appear to possess a signal peptide, an α-helical N-terminal region that contains most of the immunogenic epitopes and antigenic variability, a proline-rich domain, a choline binding domain important in anchoring the protein to the bacterial surface, and a short C-terminal tail. In one embodiment, PspAs is classified to three families and further subdivided into six clades. In one embodiment, pspAs from one serotype elicit protection against strains representing different capsule and PspA types, with the levels of protection differing among strains. In one embodiment, null expression of pspA is induced in the mutated strains described herein, by insertion of a nucleotide sequence into the chromosomeal loci encoding pspA, such as pKSD300 in another embodiment, resulting in a pspA- strain of S. pneumoniae parent.

In one embodiment, ply, referring to the gene encoding for pneumolysin, a thiol-activated toxin produced by virtually all clinical isolates of S. pneumoniae that is directly involved in pathogenesis, is mutated rendering the mutated strain unable to express ply. In another embodiment, the inability of the mutated strain to express ply, is induced by insertion duplication using the appropriate vectors, such as plasmids in one embodiment or phages in another embodiment. In one embodiment, insertion-duplication results in the inability of the mutated strain used in the compositions, cells, vaccines and methods described herein, to express cps, pspA, ply or their combination. In one embodiment, the attenuating mutation is to cps, or a double mutation to both pspA and ply, cps and ply, cps and pspA, cps, ply and pspA in other embodiments.

In one embodiment, the parent strain of S. pneumoniae strain used in the compositions, cells, vaccines and methods described herein, is TIGR4 referring to a type 4 clinical isolate, genome sequence strain, or P303 referring to a mouse virulent type 6A clinical isolate, or P1121 referring to a type 23F capsule-expressing S. pneumoniae isolate from the human nasopharynx in other embodiment. In one embodiment, the compositions, cells, vaccines and methods described herein can be used with any strain of virulent S. pneumoniae.

In one embodiment, the mutated strain derived from a parent Streptococcus pneumoniae strain, wherein said cells exhibit attenuated pathogenicity compared to those of the parent strain, and wherein the mutation is in cps, ply, pspA or their combination, which is used in the cells, compositions, vaccines or methods described herein, is in a lyophilized form. In another form, the cells, compositions, vaccines used in the methods described herein, are in the form most suitable for the administration route selected.

In one embodiment, Vaccine stabilizers used as part of the lyophilized vaccine, refer to chemical compounds added to vaccine formulations to enhance vaccine stability during periods of low temperature storage, lyophilization processing, or storage post-lyophilization. In one embodiment, the stabilizer aqueous solutions used for formulating and stabilizing the live attenuated vaccine or cells used in the vaccines or methods of the invention comprise a high molecular weigh structural additive, a disaccharide, a sugar, alcohol and water. In another embodiment, the aqueous solution also includes one or two amino acids and a buffering component. The combination of these components act in one embodiment to preserve the survival and activity of the mutated cells described herein, upon freezing and lyophilization and a long storage period subsequent to lyophilization.

In one embodiment, the mutated cells described herein, are used in the vaccines, compositions and methods described herein. In another embodiment, the invention provides a vaccine for treating, preventing or ameliorating a subject against pneumococcal infection or colonization, comprising a pharmaceutically acceptable carrier and an immunologically effective amount of mutated strain derived from a parent Streptococcus pneumoniae strain, wherein the mutation is in cps, ply, pspA or their combination.

In one embodiment, three major determinants of Streptococcus pneumoniae virulence for invasive infection, PspA, pneumolysin (ply) and cps are not a requirement for mucosal colonization raised the possibility of generating attenuated strains.

In one embodiment, capsule inhibits rather than promotes bacterial adhesion to host cells. This may be due in another embodiment, to steric hindrance by the thick polysaccharide matrix such that opaque variants expressing thicker capsules do not colonize well compared to the transparent variant of the same strain. In another embodiment, capsule do not blocks clearance by antimicrobial factors induced in the host, since the benefit of capsule is most prominent during the initial hours of colonization. In one embodiment, histologic events during early colonization are most consistent with the capsule enhancing escape of the organism from mucus and evasion of clearance by mucociliary flow. In another embodiment the optimal strategy for successful colonization requires a balance between sufficient capsular polysaccharide to escape mucus and excessive capsular polysaccharide inhibiting adherence. In one embodiment, the vaccines, compositions and methods described herein, inhibit, or prevent colonization by encapsulated strains of virulent Streptococcus pneumoniae, or Streptococcus other strains.

In another embodiment, restoration of capsule expression with negatively-charged polysaccharides has the greatest impact on the density of colonizing bacteria. In one embodiment, in pneumococcus, the negative-charged (or neutral) capsule acts to obscure the surface-oriented positively-charged quarternary amines on abundant choline residues on its structurally conserved teichoic acids. These choline residues anchor in one embodiment, a family of surface proteins required for survival in vivo, are a ligand for a host epithelial cell receptor, and are crucial for the organism's adherence and success in colonization. In another embodiment, other surface properties of the pneumococcus also serve to release it from entrapment in the mucus. These include its exoglycosidases, which in one embodiment, remove sugars found on mucus and other host molecules that bind to the organism, and in another embodiment, the IgA1 specific protease, which cleaves the. Fc_{α} linked to the mucus through secretory component. In one embodiment, unencapsulated cells derived from a virulent parent strain, wherein cps gene is not expressed, prevents subsequent colonization by the same parent strain.

In one embodiment, the vaccines described herein, or the compositions, all which are used in the methods of the invention, establish genetically-modified strains that are unable or less likely to cause disease but can colonize efficiently enough to stimulate protective immune responses.

In one embodiment, the attenuated strains described herein are cleared more efficiently compared to wild-types isolates and may, in another embodiment, be more effective at inducing protective immune responses. Encapsulation, in one embodiment, may obscure the immune response to underlying surface antigens. In another embodiment, cps mutants show limited and transient colonization that, nevertheless, is sufficient to confer significant protection. In one embodiment, colonization by cps mutants induces mucosal and systemic protection that does not depend on a response to capsular polysaccharide (serotype-independent protection).

In one embodiment, the vaccines, cells and compositions of the invention, which are used in the methods described herein, further comprising an adjuvant, cytokines, or their combination. In one embodiment, the adjuvants used in conjunction with the vaccines, or compositions described herein, in the methods described herein are mineral gels; surface active substances such as lysolecityhin; glycosides comprising saponin or, saponin derivatives such as Quil A or GP1-0100; pluronic polyols; polyanions; non-ionic block polymers, mineral oils, oil emulsions, an emulsion of vegetable oil, water and an emulsifier such as lecithin; alum, cytokines, CpG oligonucleotides, and MDP, N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine; rmLT, or AMPHIGEN and their combination. In another embodiment, the addition of another adjuvant may aid in the stimulation of a mucosal immune response. If included in one embodiment, additional adjuvants may be present in a concentration of up to about 10% by weight of the composition, with less than about 1% by weight in another embodiment. In one embodiment, the vaccines and compositions described herein, are administered as a single dose, without any adjuvants.

In one embodiment “cytokines” used in the compositions, vaccines and methods described herein, refer to small proteins secreted primarily, but not exclusively, by cells of the immune system that promote the proliferation and/or differentiative functions of other cells. Examples of cytokines include interleukins, interferons, hematopoietic colony stimulating factors (CSF), and proinflammatory factors such as tumor necrosis factor (TNF).

In one embodiment, the invention provides a method of protecting a subject against infection or colonization by a Streptococcus pneumoniae strain, comprising administering to said subject a composition comprising an immunologically effective amount of live cells of a mutated strain derived from a parent Streptococcus pneumoniae strain, wherein said cells exhibit attenuated pathogenicity compared to those of the parent strain, and wherein the mutation is in cps, ply, pspA or their combination.

In one embodiment, the cells, vaccines or compositions described hereinabove, are used in the methods of the invention. In another embodiment, the invention provides a method for preparing a mutated strain of a species of S. pneumoniae for use in a vaccine for protecting a subject against pneumococcal infection or colonization, comprising the steps of: selecting a pathogenic parent S. pneumoniae strain capable of effectively colonizing the subject's nasopharynx; deleting an entire operon of the gene encoding pneumolysin (ply), pneumococcal surface protein A (pspA), capsular polysaccharide (cps), or their combination in the selected strains, wherein the entire operon is deleted from strains that are spontaneously resistant to a predetermined antibiotic; replacing the entire operon with an operon containing the desired mutation, or double mutation; and knocking out genetic exchange, thereby preventing reversion or loss of the attenuating mutation in the mutated strain, and thereby obtaining a mutated strain, wherein said cells exhibit attenuated pathogenicity compared to those of the parent strain, and wherein said cells are capable of triggering an immune response that protects the subject against pneumococcal infection when administered as a live vaccine.

The live attenuated cells described herein are capable of triggering an immune response that protects a mammal against pneumococcal infection or colonization after one or more administrations as a live vaccine. A “protective immune response” refers in another embodiment, to any immunological response, either antibody or cell mediated immunity, or both, occurring in the mammal that either prevents or detectably reduces subsequent infection, or eliminates or detectably reduces the severity, or detectably slows the rate of progression, of one or more clinical symptoms or conditions associated with neosporosis. The term “immunologically effective amount” refers to that amount or dose of vaccine or antigen that triggers a protective immune response when administered to a mammal. In one embodiment, the protective immune response that protects the subject against pneumococcal infection or colonization is humoral, cellular or their combination.

In one embodiment, certain biological responses enhance a cell-mediated immune response whereas others preferentially enhance a humoral immune response such as stimulating an immune response in which there is an increased level of cellular compared to humoral immunity, or vice versa in another embodiment. In another embodiment, the term “humoral response” refers to an antibody-mediated immune response directed towards various regions of an antigenic determinant. In another embodiment, the term

Recombinant DNA techniques for gene replacement or gene knockout are known in the art and include, but are not limited to, those that take advantage of homologous recombination. For example, cells of a pathogenic strain of Neospora may be transformed or transfected with a vector, such as a plasmid, comprising homologous nucleotide sequences that normally flank, or are located within, for example, an essential metabolic gene, preferably a single copy gene, in a pathogenic strain of S. pneumoniae. Between or within th homologous nucleotide sequences, the vector may further comprise a nucleotide sequence that corresponds to the nucleotide sequence in the pathogenic strain but which is defective as a result, for example, of a “non-silent” change or deletion in one or more nucleotides compared to the sequence from the pathogenic strain. Transformation of a cell of the pathogenic strain with the vector is followed by integration of the defective gene sequence into the Neospora genome, which also serves to replace the original or “wild-type” sequence. Thus, the targeted gene is disabled in the transformed cell. Transformed cells may then be screened for those cells that exhibit an attenuated pathogenicity. Transformed cells exhibiting attenuated pathogenicity may then be screened again for those cells that are capable of triggering an immune response in a mammal that protects against neosporosis when administered as a live vaccine.

General techniques of genetic recombination, including vector construction, transformation, selection of transformants, host cell expression, etc., are further described in Maniatis et al, 1989, Molecular Cloning, A Laboratory Manual, Cold Spring Harbor Laboratory Press, Cold Spring Harbor, N.Y.; Ausubel et al., 1989, Current Protocols in Molecular Biology, Greene Publishing Associates & Wiley Interscience, N.Y.; Sambrook et al., 1989, Molecular Cloning: A Laboratory Manual. 2nd Ed., Cold Spring Harbor Laboratory Press, Cold Spring Harbor, N.Y.; Innis et al. (eds), 1995, PCR Strategies, Academic Press, Inc., San Diego, Calif.; and Erlich (ed), 1992, PCR Technology, Oxford University Press, New York, which are incorporated herein by reference.

In one embodiment, the term “operon”, refers to groups of bacterial genes with a common promotor, that are controlled as a unit and produce mRNA as a single piece, polycistronic messenger. In another embodiment, an operon consists of two or more structural genes, which usually code for proteins with related metabolic functions and associated control elements that regulate the transcription of the structural genes. In one embodiment, the gene whose operon is deleted in the methods described herein, for the preparation of a mutated strain, or avccines, is the gene encoding capsular polysaccharide (cps). In another embodiment, the operon is that encoding ply, pspA, cps or any combination thereof. In one embodiment, insertion/duplication methods are used to create a defined mutation having a null expression of the cps, pspA, ply genes or their combination. In one embodiment, when the mutated strain or vaccine used according to the methods described herein is intended to have cps- expression, the operon inserted is cps6A, cps7F, cps14 or cps23F capsule operons or their combination.

In one embodiment, strains that colonize efficiently and also cause invasive infection (sepsis) at high rates following intranasal colonization are being employed as the parent strain according to the methods described herein. In one embodiment, one such strain is a type 6A clinical isolate, which colonizes the nasal spaces at a density of >10⁴⁻⁵ cfu/ml for at least two weeks and causes sepsis given 10⁶ cfu. In one embodiment, mutations in either its pspA or pneumolysin (ply) gene caused no loss in fitness for colonization. In one embodiment, the step of attenuating the expression of the genes, is done because the single mutants in the type 6A background are still able to cause sepsis, therefore, a double pspA and ply mutant is constructed. The double mutant colonizes but shows in one embodiment, reduced capacity to cause sepsis after intranasal inoculation. In another embodiment, an unencapsulated mutant of the type 6A strain constructed with the Janus cassette inserted in the cps locus for PnPS biosynthesis is unable to cause sepsis despite a challenge dose of 10⁷ cfu. In another embodiment, prior exposure to live-attenuated mutants including cps, or in another embodiment, ply/pspA, or in another embodiment cps/ply, or in another embodiment their combination, decrease the density of colonization.

In another embodiment, colonization with the live-attenuated pneumococci provided herein, using the methods described herein, induce increased levels of anti-pneumococcal serum IgG (and mucosal IgA). This serum IgG response accounts in another embodiment, for the observed protection from systemic infection and in another embodiment, offers the possibility of long acting immunity. In one embodiment, the antibody-dependent effects described herein induce protection from systemic infection and in another embodiment, do not require use of a pharmacological adjuvant.

In one embodiment, the live-attenuated vaccine strains described herein, which are lacking capsular polysaccharide and the combination of Ply and PspA are both significantly attenuated and able to induce protective immunity. In another embodiment, a combination cps and ply mutations is as effective as the single cps mutation in inducing mucosal protection when provided in a two dose regimen. In another embodiment, such a step would also reduce the changes of reversion to a more virulent phenotype.

In one embodiment, the methods for preparation of mutated S. pneumoniae strains and methods for vaccine preparation described herein, further comprise the step of knocking out genetic exchange, thereby preventing reversion or loss of the attenuating mutation in the mutated strain. In one embodiment, the knocking out of genetic exchange is done by creating null expression of a competence gene, or group of competence genes. The product of the endA locus is an exported membrane-bound protein with endonuclease activity. In one embodiment, it is required for the degradation of one strand of the extracellular DNA in preparation for the translocation of the complementary strand across the bacterial membrane In one embodiment, knock out of the endA expression will inhibit genetic exchange resulting in the loss of the attenuating mutations according to the methods described herein.

In another embodiment, the comAB locus encoding a member of the family of ATP-binding cassette (ABC) proteins, which are responsible for the transport of many varied molecules, from large polypeptides to small charged molecules is knocked out. ComA is responsible in one embodiment for the transport of bacterial toxins such as hemolysin from Escherichia coli and the adenylate cyclase toxin from Bordetella pertussis. In one embodiment, ComA is associated with both the production of Act and the response of the bacteria to Act.

Another locus, recP, encodes a protein with sequence similarity to transketolases. Mutations in this determinant prevent homologous recombination of exogenous DNA but do not affect plasmid transfer. In one embodiment, the locus is deleted from the mutates cells or vaccines according to the methods described herein.

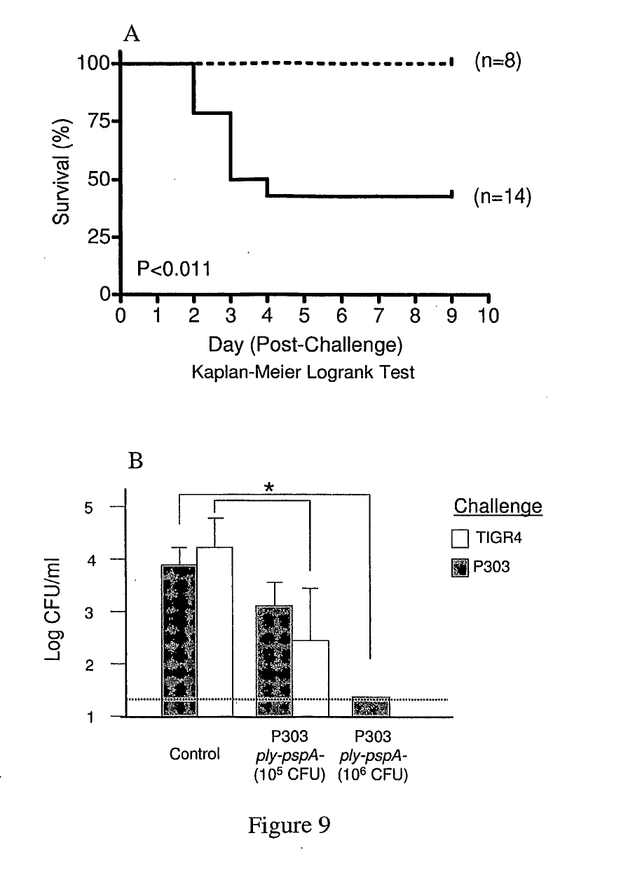

In one embodiment, the mutated strains of a species of S. pneumoniae for use in protecting a subject against pneumococcal infection or colonization, described hereinabove, are used in the vaccines and the methods for the preparation of vaccines described herein. In another embodiment, the invention provides a method for preparing a vaccine for preventing or protecting a subject against pneumococcal infection or colonization, comprising the steps of selecting a pathogenic parent S. pneumoniae strains capable of effectively colonizing the subject's nasopharynx; deleting an entire operon of the gene encoding pneumolysin (ply), pneumococcal surface protein A (pspA), capsular polysaccharide (cps), or their combination in the selected strains, wherein the entire operon is deleted from strains that are spontaneously resistant to a predetermined antibiotic; replacing the entire operon with an operon containing the desired mutation, or double mutation; knocking out genetic exchange, thereby preventing reversion or loss of the attenuating mutation in the mutated strain, and combining the cells containing the desired mutation with a pharmaceutically acceptable carrier in a form suitable for administration as a live vaccine to the subject. In one embodiment, vaccinating the subject according to the mutated cells, vaccines used in the methods described herein, results in preventing colonization of the subject by several serotypes of S. pneumoniae (see FIG. 9).

In one embodiment, the vaccines, or compositions, or mutated cells described herein, are used in the vaccination methods described herein. In another embodiment, the invention provides a method of vaccinating a subject against pneumococcal infection or colonization, comprising the step of administering to the subject an immunologically effective amount of a vaccine and a pharmaceutically acceptable carrier, wherein said vaccine comprises a mutated strain derived from a parent Streptococcus pneumoniae strain, wherein said cells exhibit attenuated pathogenicity compared to those of the parent strain, and wherein the attenuating mutation is in cps, ply, pspA or their combination, and wherein said cells are capable of triggering an immune response that protects the subject against pneumococcal infection or colonization when administered as a live vaccine. In one embodiment, the vaccination methods described herein, are administered via inhalation, or parenterally, or aerosolized.

In one embodiment, the invention provides a method of vaccinating a subject against pneumococcal disease or colonization by Streptococcus pneumoniae serotype, comprising the step of administering to the subject an immunologically effective amount of a vaccine and a pharmaceutically acceptable carrier, wherein said vaccine comprises a mutated strain derived from a parent Streptococcus pneumoniae strain, wherein said cells exhibit attenuated pathogenicity compared to those of the parent strain, and wherein the attenuating mutation is in cps, ply, pspA or their combination, and wherein said cells are capable of triggering an immune response that protects the subject against pneumococcal infection or colonization when administered as a live vaccine. In one embodiment, the vaccination methods described herein, are administered via inhalation, or parenterally, or aerosolized.

In another embodiment, the methods described herein are effective in protecting a subject against disease caused by Streptococcus pneumoniae and its various serotypes. Protecting the subject, refers in one embodiment to preventing a disease, reducing a disease severity, reducing infection; reducing pneumonia, aleviating symptoms associtaed with a disease, delaying an onset of a disease, or a combination thereof.

In one embodiment, the vaccines described herein are administered, applied, self-administered, or self-applied subcutaneously, intramuscularly, intradermally, intralymphatically, intra tumor, transdermally, intracavitarily, transbuccally, transpulmonarily, transmucosally, orally, intra nasally, intra vaginally, intra anally, intra buccally, sublingually, by inhalation.

The vaccine or mutated cells are administered parenterally, in one embodiment, either by subcutaneous or intramuscular injection. However, the vaccine may also be administered by intraperitoneal or intravenous injection, or by other routes, including orally, intransally, rectally or vaginally, and where the vaccine is so administered, a veterinarily acceptable carrier is appropriately selected. The vaccine may simply comprise attenuated mutated cells in culture fluid, which are administered directly to the subject. Alternatively, the vaccine may comprise attenuated cells combined with a pharmaceutically acceptable carrier selected from those known in the art based on the route of administration and its ability to maintain cell viability. Non-limiting examples of such carriers include water, saline, buffered vehicles and the like. Suitable other vaccine vehicles and additives are known, or will be apparent, to those skilled in the art. See, e.g., Remington's Pharmaceutical Science, 18th ed., 1990, Mack Publishing, which is incorporated herein by reference.

Vaccine regimens are selected also based on the above-described factors. Subjects may be vaccinated at any time, including just prior to or at the time of eating. Supplemental administrations, or boosters, may be required in another embodiment, for full protection. One method of detecting whether adequate immune protection is to determine seroconversion and antibody titers in the subject after vaccination. Thus, the vaccine described herein may be administered at any time during the life of a particular subject to be vaccinated, depending upon several factors, including, for example, the timing of an outbreak of pneumococcal infection among other subjects, etc. Effective vaccination may require only a primary vaccination, or a primary vaccination with one or more booster vaccinations. Booster vaccinations may be administered at any time after primary vaccination depending, for example, on the immune response after primary vaccination, the severity of the infection or colonization density, the virulence of the pneumococcal strain causing the infection, the health of the subject, etc. The timing of vaccination and the number of boosters, if any, will preferably be determined by a physician based on analysis of all relevant factors, some of which are described above.

In one embodiment, the vaccines, or mutated cells used in the vaccines and prepared according to the methods of the invention, as are described herein, are a combination vaccine, together with one or more antigens that trigger an immune response that protects a subject against a disease or a pathological condition, and a pharmaceutically acceptable carrier. In one embodiment, theses antigens are one or more antigens that trigger an immune response that protects a subject against a disease or a pathological condition is Sp 36, Sp101, Sp46, Sp91 Sp128 (Accession numbers AF291695, AF291698, AF291696, AF291697 and AF291699 respectively) or their combination.

In one embodiment, antisera recognizing Sp36, Sp91, and Sp128 also protects against lethal challenge, indicating that protection with these antigens is not capsular serotype restricted. In one embodiment, protection mediated by Sp36 extends to at least four different capsular types (types 3, 4, 6A, and 6B) of a virulent pneumococcal strain.

The following examples are presented in order to more fully illustrate the preferred embodiments of the invention. They should in no way be construed, however, as limiting the broad scope of the invention.

### EXAMPLES

### Materials and Methods

### Bacterial Strains and Culture Conditions

S. pneumoniae strains were grown as described elsewhere. Strains used in vivo were selected because of their ability to efficiently colonize the murine nasopharynx and included, TIGR4 (type 4 clinical isolate, genome sequence strain), P303 (a mouse virulent type 6A clinical isolate) and P1121 (a type 23F capsule-expressing S. pneumoniae isolate from the human nasopharynx and previously characterized in experimental human colonization). All strains were passaged intranasally in mice prior to preparation of frozen stocks.

The type 6A and 4 isolates were compared by multi-locus sequence typing (MLST) as previously described. The isolates represent two sequence types (6A ST460 and 4 ST1982), with different alleles at 7/7 loci analyzed. Thus, the type 6A and 4 isolates represent different clonal complexes as defined by eBURST (9).

The entire cps operon was deleted from spontaneously streptomycin resistant mutants (200 μg/ml) of TIGR4, P303 and D39 (type 2 clinical isolate) using the bicistronic positively and negatively selectable Janus cassette. The Janus cassette was replaced with cps6A, cps7F, cps14 or cps23F capsule operons as previous described. Encapsulation of streptomycin-resistant transformants was confirmed by positive quelling with type-specific antisera (Staten Seruminstitut, Copenhagen, Denmark). Colony opacity was visualized. All strains were passaged intranasally in mice prior to growth for preparation of frozen stocks.

A pneumolysin-negative S. pneumoniae was constructed using a previously described insertion-duplication mutant in strain D39 (type 2). Strain 6Aply was generated by transformation with chromosomal DNA from D39ply with selection for erythromycin resistance (1 μg/ml) followed by serial back transformation. Loss of pneumolysin expression was confirmed by western blotting using a monoclonal antibody to pneumolysin (Novocastra, Newcastle upon Tyne, UK).

The 6Acps/ply strain was constructed by transformation of the 6Acps strain with lysates from 6Aply with selection for erythromycin resistance as above and confirmed by western blotting.

6ApspA strain was constructed by amplifying a 1.3 kb fragment of the pspA gene from the 6A strain using the following primers LSM13: 5′-GCAAGCTTATGATATAGAAATTTGTAAC-3′(SEQ ID NO. 1), and SKH2: 5′-CCACATACCGTTTTCTTGTTTCCAGCC-3′ (SEQ ID NO. 2). The PCR product was cloned into the TOPO PCR2.1 plasmid and transformed into an E. coli TOP10F′ strain using the TOPO TA Cloning Kit (Invitrogen, Carlsbad, Calif.), and confirmed by sequencing. A 390 bp deletion was made in this 1.3 kb fragment using inverse PCR with the following primers pspa390del6AF: 5′-ACGCGTCGACGATTCAGAAGATTATGCTA-3′(SEQ ID NO. 3), and pspa390del6AR: 5′-ACGCGTCGACTCCTCTGTTGCCTTAGCTA-3′(SEQ ID NO. 4), and a spectinomycin-resistance cassette (aad9, GenBank accession number U30830) (33) was inserted into the plasmid cut with SalI. After confirmation by PCR, 6ApspA and 6Aply/pspA were generated by transformation with the plasmid DNA, with selection for spectinomycin resistance (200 μg/ml).

### Mouse Model of Nasopharyngeal Colonization

Six week-old, female C57Bl/6J (wild type) or B6.129-S2-Igh-6^{tmlCgn}/J (μMT) mice (Jackson Laboratories, Bar Harbor Me.) and B6. 129-H2-Ab1^{tmlGru}N12 (MHC II −/−) (Taconic, Germantown, N.Y.) mice were housed in accordance with Institutional Animal Care and Use Committee protocols. μMT mice contain a targeted mutation in the heavy chain locus of C57Bl/6 IgM and do not produce mature B cells or antibody. MHC II deficient mice exhibit a depletion of CD4+ T cells through the disruption of the H2-Abl gene and BALB/c mice were obtained from Taconic, Germantown N.Y. Mice were used in a previously described model of nasopharyngeal colonization with S. pneumoniae (9). Briefly, groups of at least 5 mice per condition were inoculated intranasally without anesthesia with 10 μl containing 1 to 3×10⁷ CFU of PBS-washed, mid-log phase S. pneumoniae applied to each naris. At the time indicated the animal was sacrificed, the trachea cannulated, and 200 μl of PBS instilled. Lavage fluid was collected from the nares for determination of viable counts of bacteria in serial dilutions plated on selective medium containing neomycin (20 μg/ml) to inhibit the growth of contaminants. (5 μg/ml for type 4 isolate, and 20 μg/ml for the type 6A isolate) to inhibit the growth of contaminants. The lower limit of detection for bacteria in lavage culture was 20 CFU/ml. Nasal lavage was stored at −20° C. for determination of antibody concentrations by ELISA.

S. pneumoniae Challenge

Mice where challenged intranasally with 1 to 5×10⁷ CFU of S. pneumoniae parent isolate at 5 weeks post immunization with an attenuated mutant. 5 weeks was chosen as we have previously shown that these strains are cleared from the upper respiratory tract by 4 weeks post inoculation. Where 2 dose immunization is indicated, the second dose was given 2 weeks after first dose with challenge 5 weeks after the second dose. Mice were observed for signs of sepsis over a 9 day period post challenge and animals showing signs of sepsis were euthanized and the spleen was cultured to confirm presence of pneumococci. After 9 days the remaining animals were euthanized and nasal washes were obtained for quantitative culture. The vaccine strain given up to 65 days earlier was never detected by selective plating of nasal washes, including in those obtained from immunodeficient mice. Blood was also collected from cardiac punctures and serum was stored at −20° C. for ELISA.

### Neutrophil and Complement Depletion

mAb RB6-8C5, a rat anti-mouse IgG2b directed against Ly-6G on the surface of murine myeloid (and limited subpopulations of lymphoid) lineage cells, was purified from ascites of nude mice given the RB6-8C5 hybridoma(23, 24). To deplete neutrophils, 150 μg of mAb/animal was administered by i.p. injection 24 h prior to intranasal challenge with bacteria. This dose was shown in pilot experiments to result in peripheral blood neutropenia (<50 granulocytes/μl) for a period of at least 48 h. Controls were given the equivalent i.p. dose of total rat IgG (Sigma Chemical Co., St. Louis, Mo.).

Hypocomplementemia was induced by i.p. injection of 25 μg/animal of cobra venom factor (CoVF, Quidel, San Diego, Calif.) in PBS 18 h prior to bacterial challenge. This procedure was previously shown to reduce levels of immunodetectible C3 to <3% of normal and result in a period of hypocomplementemia of >48 h

### Histology and Immunofluorescence

At the times indicated post-inoculation, the animal was sacrificed and decapitated, and the skull was fixed by 48 hr incubation in 4% paraformaldehyde in phosphate-buffered saline (PBS), and decalcified by serial incubations in 0.12 M EDTA (pH7.0), over one month before frozen in Tissue-Tek O.C.T. embedding medium (Miles) in a Tissue-Tek Cryomold. 5 μm thick sections were cut, air dried, and stored at −80° C. Frozen-imbedded tissue sections were stained with hematoxylin and eosin (H&E) following a 10 min fixation step in 10% neutral buffered formalin (NBF). Sections were then dehydrated in alcohol, cleared in xylene, and mounted in cytoseal (Richard-Allan Scientific, Kalamazoo, Mich.). For immunohistochemistry sections were post-fixed in 1:1 methanol:acetone at −20° C. for 10 minutes, followed by washing in dH₂O. Endogenous peroxidase was blocked by incubation in 2.25% H₂O₂ in dH₂O for 15 min. Sections were blocked with avidin and biotin, 15 min each, followed by a further 10 min incubation in protein blocking reagent (Coulter/Immunotech, Miami, Fla.) to prevent non-specific binding. Sections were then incubated in three steps in polyclonal rabbit anti-type 23F or anti-type 4 S. pneumoniae typing sera (Staten Serum Institute, Copenhagen, Denmark) at 4° C. overnight, 1:500 in PBT (1×PBS, 0.1% BSA, 0.2% Triton X-100), biotinylated goat anti-rabbit for 30 min, 1:200 in PBT, avidin-horse radish peroxidase (HRP) ABC reagent for 30 min (Vector Laboratories, Burlingame, Calif.), and the signal was developed using diaminobenzidine tetrahydrochloride (DAB) kit for 1-5 min (Vector Laboratories). Sections were washed with PBS after each incubation step. After the last incubation step, sections were washed with tap water, then stained with alcian blue for mucin labeling. After DAB staining the slides were fixed in 10% NBF for 3 min followed by 3 min wash in dH₂O. Sections were exposed to 3% acetic acid (pH2.5) for 3 min, before incubating with 1% alcian blue in 3% acetic acid (pH2.5) for 30 min. After washing in running water for 10 min, sections were counterstained with 0.1% Nuclear Fast Red for 40 seconds then dehydrated in alcohol, cleared in xylene, and mounted in cytoseal (Richard-Allan Scientific, Kalamazoo, Mich.). For immunofluorescent staining, tissue was post-fixed with 1:1 acetone:methanol as above, followed by blocking with peptide blocking reagent before addition of primary antibody, 1:500 in PBT. Pneumococci were detected with typing sera as above. Signal was detected with Cy3-conjugated species-specific secondary antibodies (Jackson ImmunoResearch, West Grove, Pa.) for 2 h at room temperature, 1:400 in PBT, with DAPI (4′,6-Diamidino-2-phenylindole) (Molecular Probes, Invitrogen, Carlsbad, Calif.) counterstain, 1:10000 in dH₂O. All imaging was performed on a Nikon E600 Eclipse microscope equipped with a high resolution CCD digital camera (CoolSnap CF, Roper Scientific, Tucson, Ariz.) with Nomarski optics.

### Intranasal Immunization

5-6 weeks following intranasal immunization with 10⁷ CFU of an unencapsulated mutant, 11-12 week-old female C57BL/6 mice were given an intranasal challenge with 10 μl containing 10⁷ CFU of strain P303 or TIGR4 in PBS. In pilot experiments, the dose of P303 caused sepsis within 96 h in >50% of animals. Rates of sepsis following intranasal challenge with TIGR4 were lower than other reports (<15%) because all inoculations were carried out atraumatically without anesthesia to minimize aspiration. Control mice received an intranasal dose of 10 μl of PBS. Animals were observed daily and sacrificed if they demonstrated diminished activity suggestive of sepsis. Systemic infection was confirmed by the presence of encapsulated pneumococci in cultures of blood (100 μl/animal) on selective medium. Dead animals with positive splenic cultures were considered to have succumbed from sepsis. Those animals showing no signs of septic infection during the observation period were sacrificed at 7-9 days post-challenge and the density of P303 or TIGR4 in lavage of the upper tract determined. Results were based on two independent experiments each with ≧7 mice/group.

### Measurement of IgG and IgA Levels by ELISA

Whole cell bacteria were used as the solid-phase antigen to determine the antibody titer to pneumococci. PBS washed whole bacteria, diluted with coating buffer (0.015M Na₂CO₃, 0.035M NaHCO₃) to a final OD of 0.1, were fixed by overnight incubation at 4° C. on Immulon 2HB 96 well plates (Thermo, Milford, Mass.). Plates were washed with PBS Brij (0.05%). After blocking with 1% BSA (Sigma) for 1 hour, and washing, serum, or nasal washes, in 1% BSA were added to the plate in 10-fold serial dilutions, overnight at 4° C. For serum samples, antigen specific antibodies were detected by goat anti-mouse IgG 1/4000 (heavy and light chains)-alkaline phosphatase (Sigma) for 1.5 hours, developed with pNPP (Sigma), and the absorbance at 415 nm was recorded after a standardized period of 1 hour.

For nasal wash samples a goat anti-mouse IgA was used, and developed as with serum. End-point titers were determined in triplicate by calculating the sample dilution at which the absorbance was equal to 0.1. Protein concentration in the nasal wash samples was determined by BCA™ protein assay kit (Pierce, Rockford, Ill.) to correct for variation in dilution in nasal washes and used to calculate the geometric mean titer (GMT) of antibody per μg of total protein.

### Statistical Analysis

Colonization density was expressed as the log CFU/ml for calculation of means+standard deviation. Statistical comparisons of colonization among groups were made by the non-parametric test indicated (GraphPad Prism 4).

### Example 1

### Unencapsulated Mutants Colonize the Nasal Spaces

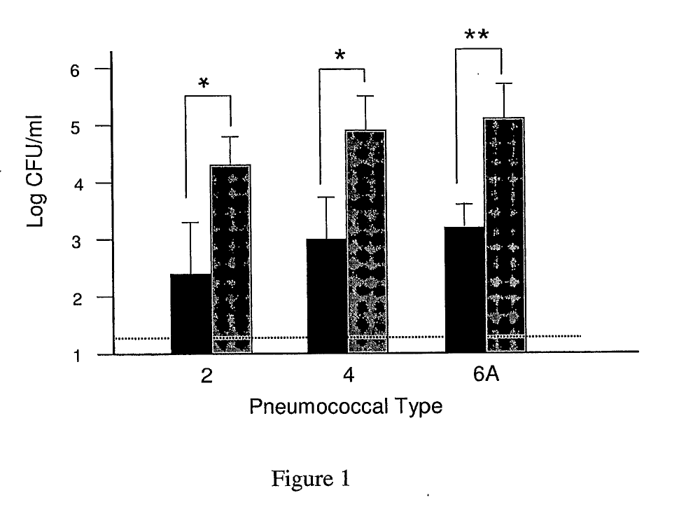

The contribution of capsule during colonization was assessed by comparing encapsulated isolates with their isogenic unencapsulated mutants in a murine model of colonization following intranasal inoculation. Mutants lacking cps were generated by use of Janus cassette technology that allows for construction of unmarked, in-frame deletions. TIGR4cps- consistently colonized C57BL/6 mice but at a density 10 to 100-fold less compared to its parent strain in quantitative culture of upper airway lavages at 2 d post-inoculation (FIG. 1). A similar contribution of encapsulation to colonization was also demonstrated by comparison of isolates of other types (2 and 6A) with and without cps.

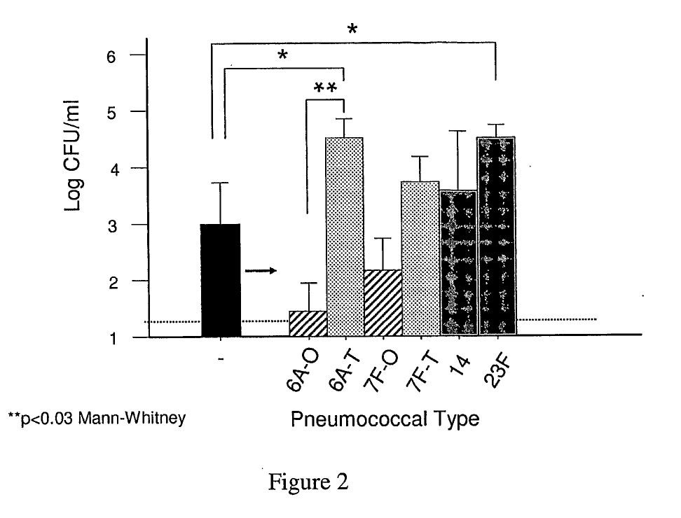

To confirm that the decrease in fitness for colonization was due to the loss of the capsule, the deletion of cps in TIGR4 was corrected by insertion of the cps locus derived from heterologous pneumococcal types. Correction of capsule expression was sufficient to restore the density of colonization as assessed at 2 d post-inoculation to wild-type levels (FIG. 2). However, in these constructs with same genetic background, the contribution of encapsulation depended on the capsule type with the greatest effect for negatively charged types, 6A and 23F, and lesser effect for types with neutral charge, 7F and 14. An additional factor affecting colonization was the thickness of the capsule expressed by transformants. The more mucoid opaque variants with increased amounts of capsular polysaccharide showed levels of colonization below that of the unencapsulated mutant whereas less mucoid, transparent variants with thinner zones of capsule colonized relatively efficiently.

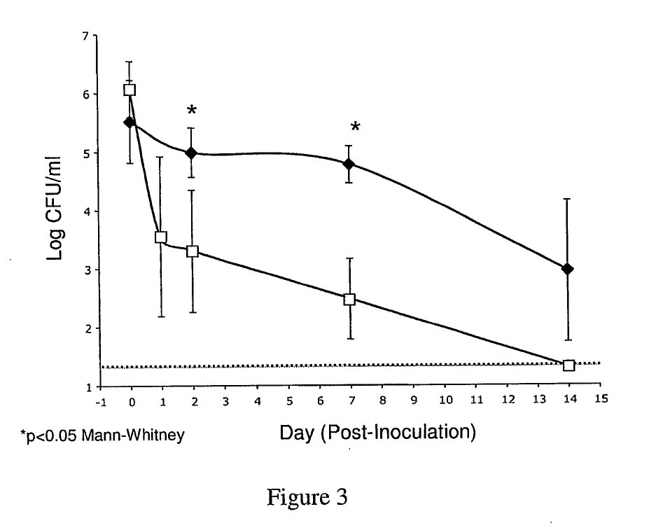

To determine when encapsulation affects colonization, TIGR4 and TIGRcps- were compared over the period during which pneumococci could be recovered from upper airway lavages. TIGRcps- was able to persist for up to 7 d post-inoculation (FIG. 3). There was, however, a marked decline in colonization density between 1 and 20 h post-inoculation for TIGR4 cps- that was not seen for TIGR4, which showed a more gradually decrease over 14 d post-inoculation. After this initial decline for TIGR4 cps-, the rate of decrease in the density of colonizing bacteria was similar regardless of the expression of capsule. During the period from 2 to 14 d post-inoculation, the density of TIGR4 cps-averaged >50-fold less compared to the isogenic encapsulated parent strain at the same time point. Together these results showed that capsule is not necessary but may enhance colonization, particularly during initial events in the host, with the extent of its contribution dependent on its composition and amount.

### Example 2

### Capsule does not Impact on Opsonophagocytosis Clearance During Colonization

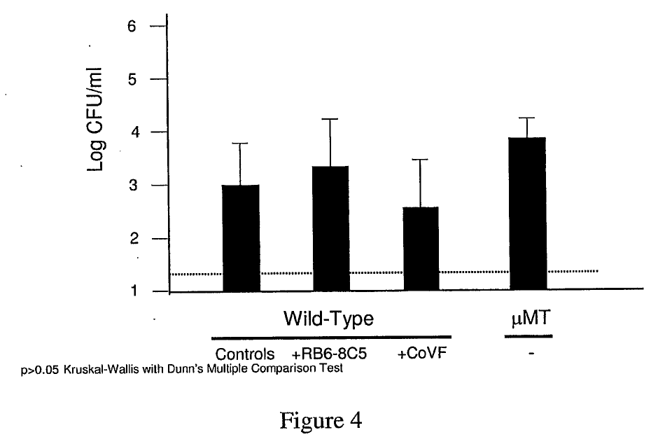

Histological examination of colonized nasal tissues confirmed that colonizing pneumococci induce neutrophil influx into lateral nasal spaces by 1 d with a maximal response by 3 d post-inoculation. Immunoflourescent staining of frozen tissue to detect bacteria demonstrates that these dense clusters of neutrophils have engulfed pneumococci, but that not all pneumococci become associated with neutrophils (data not shown). To test whether neutrophil-mediated clearance accounted for the lower density of colonization by unencapsulated pneumococci, mice were treated with RB6-8C5, a rat mAB to murine Ly6.G prior to intranasal challenge(23). This effectively depleted neutrophils from peripheral blood and in tissue sections of colonized mice (data not shown). It was predicted that this treatment would decrease opsonophagocytic clearance and allow for enhanced colonization by unencapsulated pneumococci. However, there was no significant effect of RB6-8C5 treatment compared to controls on the density of TIGR4 cps- at 2 d post-inoculation (FIG. 4). There was also no effect of neutrophil depletion on the colonization density of the encapsulated parent strain, although RB6-8C5 treatment made it more likely these animals developed bacteremic infection (7/8 mice treated with RB6-8C5 mice v. ⅛ in controls). Similarly, no effect on the colonization density of TIGR4 cps- was observed following complement depletion by systemic administration of cobra venum factor prior to nasal challenge or in genetically-modified congenic mice (μMT) that fail to generate specific antibody. These findings indicated that neutrophil-, complement- or antibody-mediated clearance mechanisms do not contribute substantially to the lower density of colonization by unencapsulated mutants. These observations left in question the role of capsule during colonization. A further conclusion was that the acute neutrophil influx that occurs soon after acquisition of the organism is insufficient for clearance from the mucosal surface.

### Example 3

### Effect of Capsule on the Dynamics of Colonization

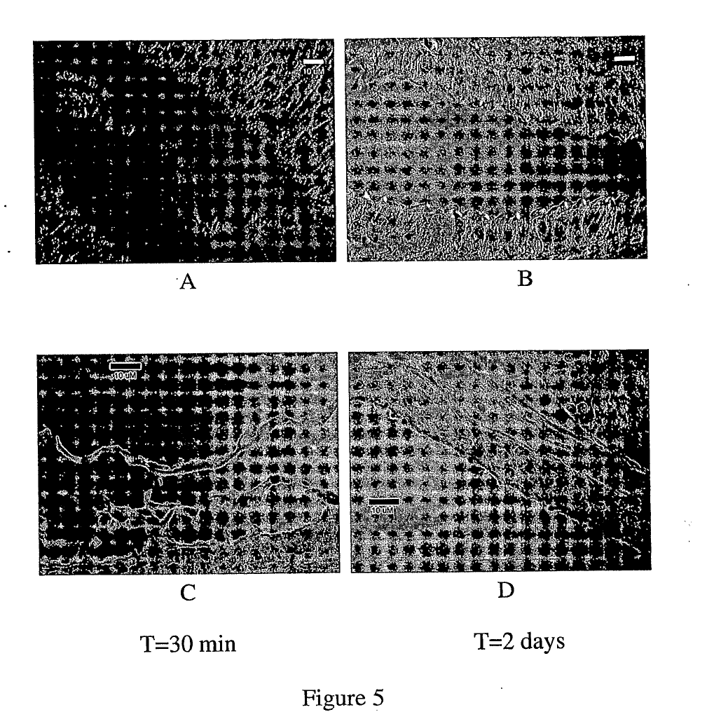

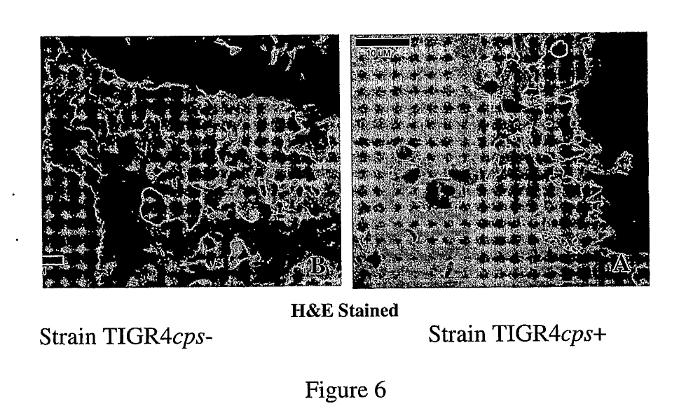

To define the role of capsule in pneumococcal colonization, the events during the initial 2d period post-inoculation, during which the majority of the deficit in colonization by unencapsulated mutants develops, were visualized in tissue sections. For TIGR4, initially (time=30 min) bacteria are confined to the lumen of nasal spaces were they associate with amorphous material (FIG. 5A). This luminal material is mucus based on its staining with alcian blue that identifies acidic mucopolysaccharides (FIG. 5C). By 2d post-inoculation, these encapsulated pneumococci had transited to the mucosal surfaces where they were found in the thin mucus layer overlying epithelial cells (FIG. 5B, D). At later time points up to 14 d, pneumococci remained in the mucus layer over the epithelial cells indicating that this was the site of stable colonization. Unencapsulated mutants were also seen initially in the luminal mucus (T=30 minutes), but unlike the encapsulated parent were heavily agglutinated (FIG. 6 A v. B). When rare unencapsulated mutants were seen later at 20 h post-inoculation these were still associated with luminal mucus rather than along the epithelial surface suggesting an inability to transit to the epithelial surface as seen for the encapsulated parent (FIG. 6C v. D). These findings suggest that the capsule acts to inhibit association with luminal mucus that promotes clearance in the early phases of colonization. Encapsulated pneumococci appeared to be more likely to escape from this material to established adherence along the mucosal surface where direct interaction with underlying epithelial cells was possible.

### Example 4

### Unencapsulated Mutants as a Live, Attenuated Vaccine

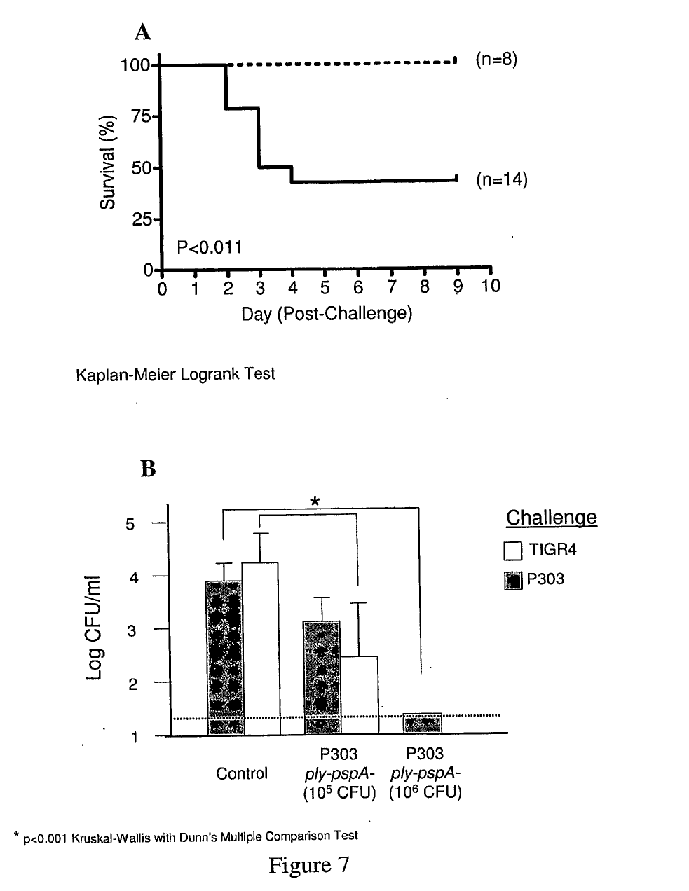

The finding that unencapsulated mutants could still colonize once they evaded early clearance by agglutination with lumenal mucus, indicate that these mutants could provide a safe means to stimulate protective immunity. To test this hypothesis, a type 6A isolate that is virulent in mice following intranasal inoculation, P303, was used to challenge previously colonized animals. 10⁷ CFU of an unencapsulated mutant, P303 cps-, or vehicle control was applied intranasally without adjuvant and the mice were allowed to clear their carrier state over 5-6 weeks. Following challenge with a lethal dose (10⁷ CFU) of the encapsulated parent, previously colonized mice were protected from sepsis (FIG. 7A). In addition, mice previously colonized with P303 cps- showed diminished levels of colonization by P303 at 9 d post-challenge (FIG. 7B). Likewise, there was reduced colonization by TIGR4 in mice previously colonized with P303cps- compared to mice previously innoculated with vehicle control. This confirmed that protection from colonization could be independent of the immune response to capsular polysaccharide. An additional conclusion was that attenuated strains of the present invention induce, under the conditions utilized herein, non-strain specific protective immunity. In another embodiment, attenuated strains of the present invention induce, under the conditions utilized herein, protective immunity not specific to a particular steptococcal type. As anticipated, this unencapsulated mutant was completely attenuated as none of the animals given P303 cps- developed signs of infection. Thus, a single dose of this live, attenuated mutant without adjuvant was sufficient to provide both mucosal and systemic protection from an otherwise fatal respiratory challenge that mimics the route of natural infection

### Example 5

### Attenuating Mutations to Enable Vaccination with a Single Dose and without an Adjuvant

The results show that pneumococci lacking cps (FIG. 1) or ply and pspA retain the ability to colonize the mouse nasopharynx. Next, the data shows that colonization with a cps mutant followed 6 weeks later by intranasal challenge with the encapsulated virulent parent strain (P303) protects against sepsis (FIG. 7) and colonization (FIG. 9). Moreover, there was significant cross protection from colonization when the immunized mice were challenged with an unrelated strain of another serotype. This demonstrates that protection is not serotype dependent as is the case for current pneumococcal vaccines. In another embodiment, attenuated strains of the present invention induce, under the conditions utilized herein, protective immunity not specific to a particular steptococcal group. Likewise, immunization with a ply-, pspA- double mutant conferred complete protection from sepsis (FIG. 9) as well as recolonization. The ply-, pspA double mutant also conferred protection from colonization by an unrelated strain. Note that in contrast to current pneumococcal vaccines, protection was achieved using a single dose without the need for an additional adjuvant.

### Example 6

### Evaluation of Attenuation and Colonization of Vaccine Strains

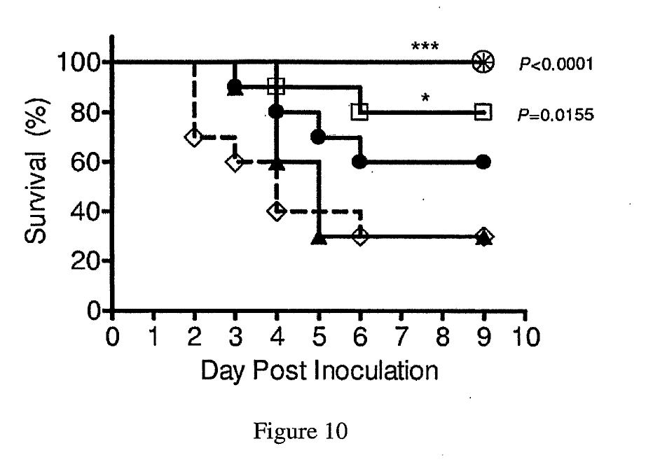

To generate live-attenuated vaccine candidates, genes of each of the three major virulence determinants of S. pneumoniae (cps, ply, pspA) were independently interrupted in a type 6A isolate capable of inducing sepsis following intranasal challenge. To establish which strains were attenuated in the mouse model, the survival of C57Bl/6 mice was followed after a high dose of these mutant strains (10⁷ CFU/mouse) given intranasally. The 6Aply mutant remained as virulent as the parent strain, while 6ApspA displayed partial attenuation and 6Acps was completely avirulent (FIG. 10). Because of the limited attenuation of the vaccine strains lacking the proteinaceous virulence determinants, a double mutant was constructed (6Aply/pspA) and was significantly, but still not completely attenuated.

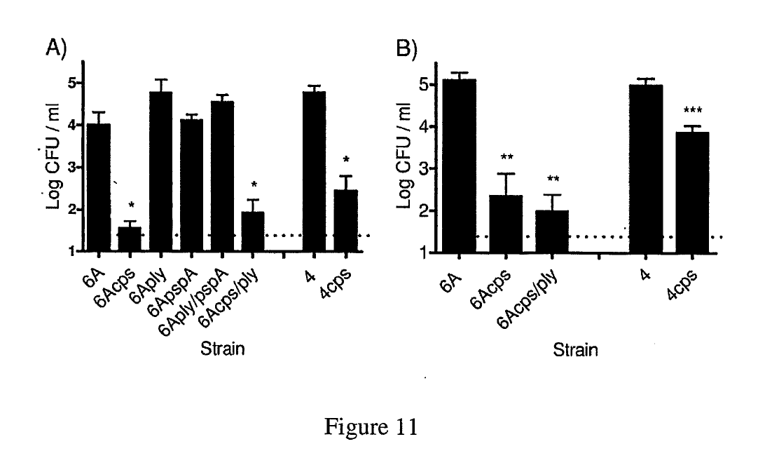

Since the ability of live vaccine strains to induce protection would be enhanced by their persistence on the mucosal surface, the effect of attenuating mutations was determined by comparing quantitative colonization of the upper respiratory tract. At day 9 post inoculation the 6Aply, 6ApspA and 6Aply/pspA mutants displayed similar colonization density compared to the parent strain (FIG. 11A). In contrast colonization by the cps mutants were not significantly above the limit of detection by day 9. A similar result was seen with a cps mutant in a type 4 isolate. To further investigate the unencapsulated mutants, the density of colonization was also determined at day 2 post-inoculation, and the cps mutants of both serotypes demonstrated colonization albeit at a reduced density compared to the corresponding parent strains (FIG. 11B).

### Example 7

### Protection and Cross Protection of Vaccine Strains

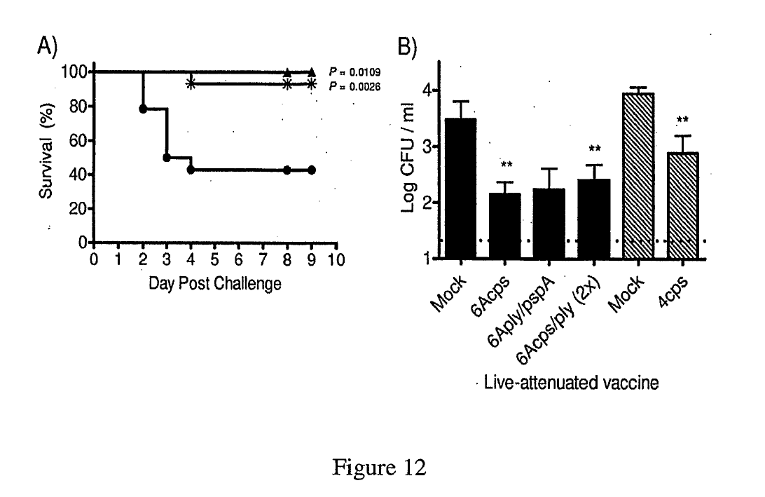

The ability to protect against sepsis was assessed for the vaccine strains that showed the greatest attenuation (6Acps and ply/pspA). Colonization by live-attenuated vaccine strains was used to immunize mice, and 5 weeks post-immunization the previously colonized mice were challenged intranasally with a high dose of the parent strain (10⁷ CFU/mouse). Both 6Acps and double mutant (6Aply/pspA) vaccine strains induced significant protection (FIG. 12A).

Mucosal protection induced by prior colonization by vaccine strains was also assessed. The density of colonization of challenge strains was determined 9 days post inoculation with the parent isolate. Following challenge with either the type 4 or 6A parent isolate the density of colonization was significantly reduced for mice that were immunized with the corresponding cps vaccine strains (FIG. 12B). Combinations of mutations in 6A vaccine strains, including ply/pspA and cps/ply, induced mucosal protection against the 6A parent isolate but for the later mutant a significant effect was only seen after two doses of the live-attenuated vaccine. These data show that the attenuated vaccine strains can induce mucosal and systemic protection.

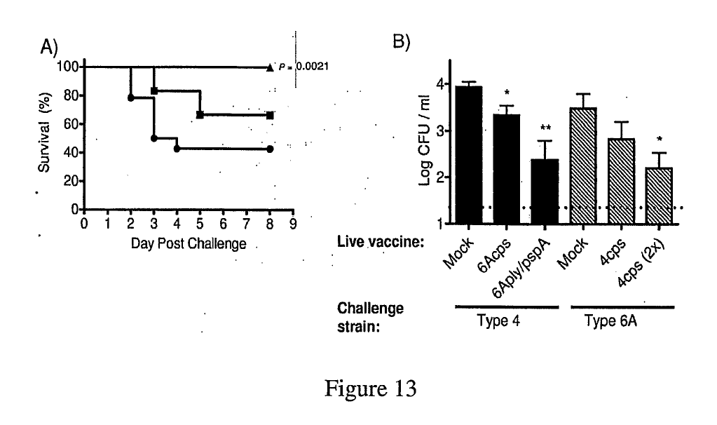

The cps mutant was able to protect significantly from sepsis, and also showed the greatest mucosal protection, therefore a type 4 cps mutant was used to assess whether immunity induced by one strain could protect against another (cross protection). Colonization of the type 4 cps mutant was used to immunize mice before challenge with a high dose of the 6A isolate (10⁷ CFU/mouse). With a single dose of the type 4 cps vaccine strain mice may have partial protection from sepsis, however with two doses of this vaccine strain there was complete protection from sepsis with the type 6A challenge (FIG. 13A). Again the mucosal protection was investigated. A single dose of type 4 cps mutant partially reduced colonization by the 6A isolate, with further reduction following a second dose (FIG. 113B). Single dose 6A vaccine strains (cps, ply/pspA) were also used to investigate cross protection from colonization with a high dose type 4 challenge. This type 4 isolate causes septic infection at a low rate in our mouse model, so mucosal not systemic protection was evaluated. Both vaccine strains were proficient at reducing colonization by a distantly related isolate. These results show that the live-attenuated strains are able to elicit protective immunity that is serotype-independent.

### Example 8

### Mechanism of Protection of Vaccine Strains

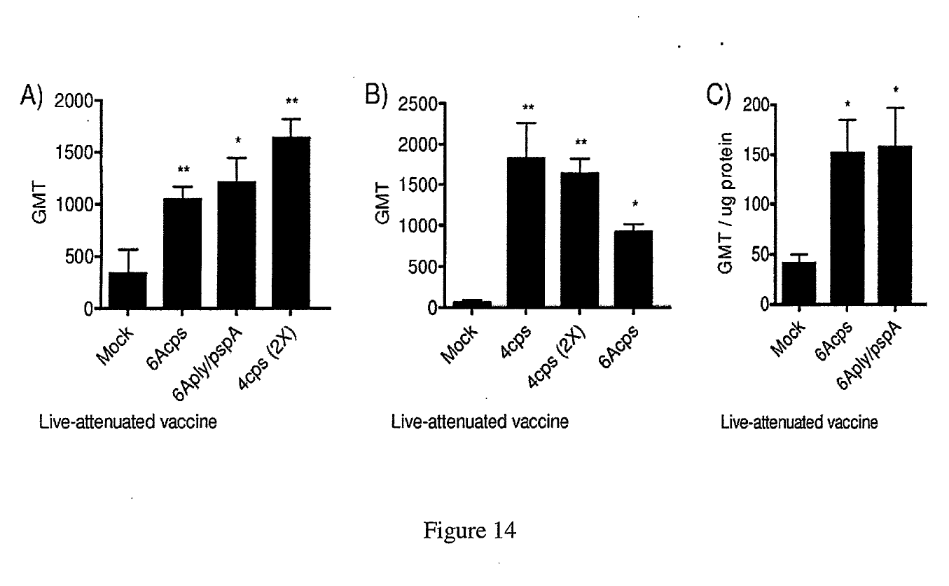

The role of humoral and cell mediated immunity was investigated to determine the mechanism of protection from the live-attenuated vaccine strains. To assess the humoral immune response, the IgG titers to whole pneumococci were determined in serum for groups in which there was protection from either a type 6A or 4 challenge (FIGS. 14A and B respectively). The levels of pneumococcal specific IgG in serum were significantly increased after immunization with vaccine strains (cps, ply/pspA). The levels of IgA were then quantified in nasal wash samples to determine the antibody titer on the mucosal surface. These values were corrected for variation in dilution of nasal wash samples by determination of total protein. The mice immunized with vaccine strains displayed significant increases in IgA titers (FIG. 14C).

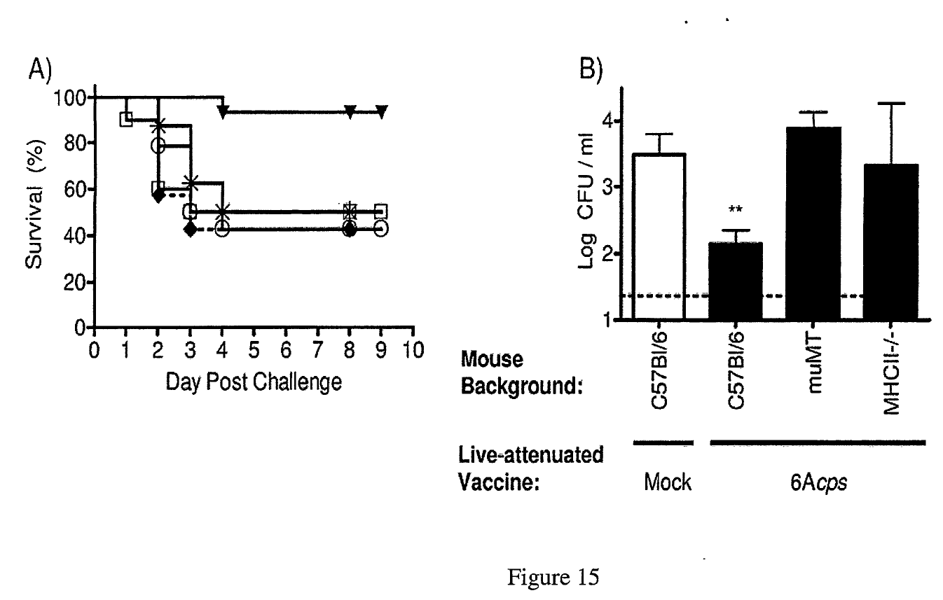

Having demonstrated an increase in antibody titer due to immunization, it was then investigated if protection from sepsis is dependent on humoral or cell mediated immunity. μMT mice (which are unable to generate specific antibody) and MHCII^{-/-} (exhibiting a depletion of CD4⁺ T cells) were immunized with the 6Acps vaccine strain to see if protection was sustained. Neither the μMT nor the MHCII^{-/-} mice were protected against challenge with the type 6A isolate (FIG. 15A). This indicates that the protection from sepsis is antibody dependent and requires CD4⁺ T cells. Antibody dependence in mucosal protection was also investigated. The reduction in colonization seen with the wild type C57Bl/6 mice following immunization was not seen with the μMT nor the MHCII^{-/-} mice (FIG. 15B). This suggests that mucosal protection following immunization is also antibody dependent and requires CD4⁺ T cells.

All references cited in this specification either supra or infra are hereby incorporated by reference. The discussion of the references herein is intended merely to summarize the assertions made by their authors and no admission is made as to the accuracy or pertinency of the references or that any reference is material to patentability.

From the foregoing, it will be appreciated that, although specific embodiments of the invention have been described herein for purposes of illustration, various modifications may be made without deviating from the spirit and scope of the invention.

What is claimed is:

1. A mutated strain derived from a parent Streptococcus pneumoniae strain, wherein said cells exhibit attenuated pathogenicity compared to those of the parent strain, and wherein the mutation is in cps, ply, pspA or their combination.

2. The mutated strain of claim 1, wherein the mutated strain contains an attenuating mutation to genes encoding capsular polysaccharide (cps).

3. The mutated strain of claim 1, containing a double attenuating mutation, wherein the double mutation is to the genes encoding pneumolysin (ply) and pneumococcal surface protein A (pspA).

4. The mutated strain of claims 2 or 3, wherein the attenuating mutation results in null expression of the Streptococcus pneumoniae gene or genes.

5. The mutated strain of claim 1, wherein the parent S. pneumoniae strain is TIGR4 (type 4 clinical isolate, genome sequence strain), P303 (a mouse virulent type 6A clinical isolate), or P1121 (a type 23F capsule-expressing S. pneumoniae isolate from the human nasopharynx).

6. The mutated strain of claim 1, in a lyophilized form.

7. A vaccine for treating, preventing or ameliorating a subject against pneumococcal infection or colonization, comprising a pharmaceutically acceptable carrier and an immunologically effective amount of mutated strain derived from a parent Streptococcus pneumoniae strain, wherein the mutation is in cps, ply, pspA or their combination.

8. The vaccine of claim 7, wherein the mutated strain is incapable of forming a polysaccharide capsule.

9. The vaccine of claim 7, wherein the mutated strain contains an attenuating mutation to a gene encoding capsular polysaccharide (cps).

10. The vaccine of claim 7, containing a double attenuating mutation, wherein the double mutation is to the genes encoding pneumolysin (ply) and pneumococcal surface protein A (pspA). I would do separate dependent claims to these as well.

11. The vaccine of claims 9 or 10, wherein the attenuating mutation results in null expression of the Streptococcus pneumoniae gene or genes.

12. The vaccine of claim 7, wherein the parent S. pneumoniae strain is TIGR4 (type 4 clinical isolate, genome sequence strain), P303 (a mouse virulent type 6A clinical isolate), or P1121 (a type 23F capsule-expressing S. pneumoniae isolate from the human nasopharynx).

13. The vaccine of claim 7, further comprising an adjuvant, cytokines, or their combination.

14. The vaccine of claim 13, wherein the adjuvant is an oil-in-water emulsion.

15. The vaccine of claim 7, in a lyophilized form.

16. A method of protecting a subject against disease or colonization by a Streptococcus pneumoniae strain, comprising administering to said subject a composition comprising an immunologically effective amount of live cells of a mutated strain derived from a parent Streptococcus pneumoniae strain, wherein said cells exhibit attenuated pathogenicity compared to those of the parent strain, and wherein the mutation is in cps, ply, pspA or their combination.

17. The method of claim 16, wherein the mutated strain is incapable of forming a polysaccharide capsule.

18. The method of claim 16, wherein protecting a subject against disease or colonization by a Streptococcus pneumoniae strain, comprises preventing a disease, reducing a disease severity, reducing infection; reducing pneumonia, aleviating symptoms associtaed with a disease, delaying an onset of a disease, or a combination thereof.

19. A method for preparing a mutated strain of a species of S. pneumoniae for use in a vaccine for protecting a subject against pneumococcal infection or colonization, comprising the steps of: selecting a pathogenic parent S. pneumoniae strain capable of effectively colonizing the subject's nasopharynx; deleting an entire operon of the gene encoding pneumolysin (ply), pneumococcal surface protein A (pspA), capsular polysaccharide (cps), or their combination in the selected strains, wherein the entire operon is deleted from strains that are spontaneously resistant to a predetermined antibiotic; replacing the entire operon with an operon containing the desired mutation, or double mutation; and knocking out genetic exchange, thereby preventing reversion or loss of the attenuating mutation in the mutated strain, and thereby obtaining a mutated strain, wherein said cells exhibit attenuated pathogenicity compared to those of the parent strain, and wherein said cells are capable of triggering an immune response that protects the subject against pneumococcal infection when administered as a live vaccine.

20. The method of claim 19, wherein the gene whose operon is deleted is the gene encoding capsular polysaccharide (cps).

21. The method of claim 20, wherein the operon containing the desired mutation is cps6A, cps7F, cps14 or cps23F capsule operons or their combination.

22. The method of claim 17, wherein the pathogenic parent S. pneumoniae strain is TIGR4 (type 4 clinical isolate, genome sequence strain), P303 (a mouse virulent type 6A clinical isolate), or P1121 (a type 23F capsule-expressing S. pneumoniae isolate from the human nasopharynx).

23. The method of claim 17, the pathogenic parent S. pneumoniae strain is P303 (a mouse virulent type 6A clinical isolate).

24. The method of claim 17, wherein the genes whose operons are deleted are the gene encoding pneumolysin (ply) and pneumococcal surface protein A (pspA).

25. The method of claim 24, wherein the operons containing the desired mutation result in null expression of ply, pspA or their combination.

26. A method for preparing a vaccine for preventing or protecting a subject against pneumococcal infection or colonization, comprising the steps of selecting a pathogenic parent S. pneumoniae strains capable of effectively colonizing the subject's nasopharynx; deleting an entire operon of the gene encoding pneumolysin (ply), pneumococcal surface protein A (pspA), capsular polysaccharide (cps), or their combination in the selected strains, wherein the entire operon is deleted from strains that are spontaneously resistant to a predetermined antibiotic; replacing the entire operon with an operon containing the desired mutation, or double mutation; knocking out genetic exchange, thereby preventing reversion or loss of the attenuating mutation in the mutated strain, and combining the cells containing the desired mutation with a pharmaceutically acceptable carrier in a form suitable for administration as a live vaccine to the subject.

27. The method of claim 26, wherein the gene whose operon is deleted is the gene encoding capsular polysaccharide (cps).

28. The method of claim 27, wherein the operon containing the desired mutation is cps6A, cps7F, cps14 or cps26F capsule operons.

29. The method of claim 26, wherein the pathogenic parent S. pneumoniae strain is TIGR4 (type 4 clinical isolate, genome sequence strain), P303 (a mouse virulent type 6A clinical isolate), or P1121 (a type 26F capsule-expressing S. pneumoniae isolate from the human nasopharynx).

30. The method of claim 26, the pathogenic parent S. pneumoniae strain is P303 (a mouse virulent type 6A clinical isolate).

31. The method of claim 26, wherein the genes whose operons are deleted are the gene encoding pneumolysin (ply) and pneumococcal surface protein A (pspA).

32. The method of claim 26, wherein the operons containing the desired mutation result in null expression of ply, pspA or their combination.

33. A method of vaccinating a subject against pneumococcal infection or colonization, comprising the step of administering to the subject an immunologically effective amount of a vaccine and a pharmaceutically acceptable carrier, whereby said vaccine comprises a mutated strain derived from a parent Streptococcus pneumoniae strain, wherein said cells exhibit attenuated pathogenicity compared to those of the parent strain, and wherein the attenuating mutation is in cps, ply, pspA or their combination, and wherein said cells are capable of triggering an immune response that protects the subject against pneumococcal infection or colonization when administered as a live vaccine.

34. The method of claim 33, whereby the isolated strain is incapable of forming a polysaccharide capsule.

35. The method of claim 33, whereby the mutated strain, which contains attenuating mutation to genes encoding capsular polysaccharide (cps).

36. The method of claim 33, whereby the mutated strain, which contains a double attenuating mutation, whereby the double attenuating mutation is to the genes encoding pneumolysin (ply) and pneumococcal surface protein A (pspA).

37. The method of claim 33, whereby the pathogenic parent S. pneumoniae strain is TIGR4 (type 4 clinical isolate, genome sequence strain), P303 (a mouse virulent type 6A clinical isolate), or P1121 (a type 23F capsule-expressing S. pneumoniae isolate from the human nasopharynx).

38. The method of claim 33, whereby the vaccine further comprises an adjuvant cytokines, or their combination.

39. The method of claim 33, whereby the adjuvant is an oil-in-water emulsion.

40. The method of claim 33, whereby the vaccine is effective against an unrelated strain of another serotype of Streptococcus.

41. The method of claim 33, whereby the vaccine is in a lyophilized, an aerosolized, or a parenteral form.

42. The method of claim 33, whereby the step of administering is done via inhalation.

43. The method of claim 33, whereby the immune response that protects the subject against pneumococcal infection or colonization is humoral, cellular or their combination.

44. A combination vaccine, comprising the vaccine of claim 7, together with one or more antigens that trigger an immune response that protects a subject against a disease or a pathological condition, and a pharmaceutically acceptable carrier.

45. The vaccine of claim 44, wherein the one or more antigens other than a S. pneumoniae antigen that trigger an immune response that protects a subject against a disease or a pathological condition is Sp 36, Sp101, Sp46, Sp91 Sp128 (Accession numbers AF291695, AF291698, AF291696, AF291697 and AF291699 respectively) or their combination.


SEQUENCE LISTING (4 sequences)

SEQ ID NO: 1 - DNA, 28 bp, Streptococcus pneumoniae
gcaagcttatgatatagaaatttgtaac

SEQ ID NO: 2 - DNA, 27 bp, Streptococcus pneumoniae
ccacataccgttttcttgtttccagcc

SEQ ID NO: 3 - DNA, 29 bp, Streptococcus pneumoniae
acgcgtcgacgattcagaagattatgcta

SEQ ID NO: 4 - DNA, 29 bp, Streptococcus pneumoniae
acgcgtcgactcctctgttgccttagcta